(19) United States
(12) Patent Application Publication
(10) Pub. No.: US 2014/0113247 A1
(43) Pub. Date: Apr. 24, 2014

(54) FIXTURES FOR DENTAL IMPLANTS

(71) Applicant: AETON MEDICAL LLC, (US)

(72) Inventor: Roger S. RANCK, Ambler, PA (US)

(73) Assignee: AETON MEDICAL LLC, Philadelphia, PA (US)

(21) Appl. No.: 13/874,976

(22) Filed: May 1, 2013

Related U.S. Application Data
(63) Continuation of application No. 12/332,524, filed on Dec. 11, 2008, now abandoned.

(51) Int. Cl. A61C 8/00; A61K 6/083

(52) U.S. Cl. CPC A61C 8/008; A61C 8/0012; A61K 6/0835; A61C 8/0069; A61C 8/0054
USPC 433/173

(57) ABSTRACT

A temporary coping for placement as an intermediate structure between a temporary restorative tooth and a support on a dental implant may comprise a coronal end, an apical end, and a lateral wall surrounding a hollow interior. The hollow interior may be dimensioned to receive a support mountable on a dental implant to place the temporary coping in mating engagement with the support along a substantial length of the hollow interior. The temporary coping may be made of a dental restoration material chosen from bis-acrylic and a bis-acrylic composite and is chemically bondable with the temporary restorative tooth.

### TECHNICAL FIELD

The present disclosure relates generally to fixtures for dental implants. In particular, the present disclosure relates to temporary copings that are configured to form a bond with dental restorations.

### BACKGROUND

Since the discovery that titanium can fuse to bone, titanium dental implants have represented a growing field of dental reconstruction technology for replacing natural teeth. During implantation, a hole is drilled through the gingiva, the gums surrounding the root of a tooth, and into the jawbone. A titanium or titanium alloy screw is then fixed within the hole of the jawbone. Over a period, for example months, the titanium screw fuses to the jawbone through a process called osseointegration.

During the time required for the titanium screw to fuse to the jawbone, a temporary tooth is required that allows the gingiva to heal and properly form around the restorative tooth. The restorative tooth typically secures to an abutment or provisional coping that attaches to the titanium screw. More specifically, a temporary coping is affixed to the abutment or titanium provisional coping, and an outer, restorative tooth is formed over the temporary coping. The temporary coping acts as an intermediate fixture between the abutment or the titanium provisional coping, and the restorative tooth and allows the restorative tooth to be fabricated outside of the patient's mouth. This system is known as a temporary restoration.

The temporary coping is generally frustoconical in shape and has a hollow interior to mate to a provisional coping or an abutment. During the fitting of the temporary restoration, the temporary coping is positioned on the provisional coping or abutment and measured to determine the appropriate size of the temporary coping. The temporary coping is then modified as necessary, such as, for example, by trimming the top of the temporary coping to fit. The temporary coping is then removed and the restorative tooth is placed on the temporary coping prior to attaching the temporary coping with the restorative tooth onto the abutment. Once the gums have healed around the implant and temporary restoration, the temporary restoration is removed and a permanent replacement tooth is attached to the implant.

Conventional temporary copings have been made of materials such as, for example, nylon, polyethyl ethyl ketone (PEEK), and polymethyl methacrylate (PMMA). Restorative teeth, also called caps, shells, or crowns, are typically formed of materials including composite or plastic, such as nylon or acrylic. It is desirable that the temporary coping and temporary restorative tooth comprise material(s) capable of lasting an extended period of time, and be able to withstand the mastication forces exerted on the temporary restoration. Thus, there is a need in the industry for materials that have enough strength to be able to withstand such mastication forces for an extended period of time.

Conventional processes for securing a restorative tooth on a temporary coping rely on mechanical bonds. Conventional temporary copings may have grooves. retention rings, surface texturing, or other physical features provided on an external surface of the coping to create a mechanical bond between the temporary coping and the restorative tooth. Additionally, adhesives such as, for example, cement are used to bond the tooth to the temporary coping. The bond between the restorative tooth and the temporary coping is a common source of failure for dental restorations. Thus, there is a long-felt need in the industry for an improved bond between the restorative tooth and the temporary coping.

Although the present invention may obviate one or more of the above-mentioned disadvantages, it should be understood that some aspects of the invention might not necessarily obviate one or more of those disadvantages.

### SUMMARY

In accordance with various exemplary embodiments, the present teachings may provide a temporary coping comprising a material capable of forming a chemical bond with a restorative tooth. Further exemplary embodiments of the present teachings provide a method for bonding a temporary coping to a restorative tooth, the method comprising forming a restorative tooth on a temporary coping and curing the restorative tooth to form the chemical bond between a material of the temporary coping and a material restorative tooth.

As described herein, the present teachings relate to temporary copings and methods of bonding a restorative tooth to a temporary coping.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the present teachings or claims.

### BRIEF DESCRIPTION OF THE DRAWINGS

The following figures, which are incorporated in and constitute a part of the specification, serve to further illustrate exemplary embodiments of the invention. The figures are not, however, intended to be restrictive of the invention or claims. In the drawings,

FIG. 1 is a perspective side view of a temporary coping according to an exemplary embodiment of the present disclosure;

FIG. 2 is a perspective side view showing an internal portion of a restorative tooth bonded to the exemplary temporary coping of FIG. 1;

FIG. 3 is a perspective side view of a temporary coping according to an exemplary embodiment of the present disclosure;

FIG. 4 is a perspective side view showing an internal portion of a restorative tooth bonded to the exemplary temporary coping of FIG. 3;

FIG. 5 is a perspective side view of a provisional coping according to an exemplary embodiment of the present disclosure; and

FIG. 6 is a cutaway view of a temporary coping according to an exemplary embodiment of the present disclosure.

### DESCRIPTION OF EXEMPLARY EMBODIMENTS

Reference will now be made to various exemplary embodiments illustrated in the accompanying figures. However, these various exemplary embodiments are not intended to limit the disclosure. To the contrary, the disclosure is intended to cover alternatives, modifications, and equivalents.

The term “improved bond” and variants thereof, as used herein, means an increase in resistance to fracture, an increase in resistance to crack propagation when a crack is present, and/or a decrease in the likelihood of bond failure between the temporary coping and the restorative tooth as compared to conventional mechanical bonds, including adhesive bonding agents such as cement, between a temporary coping and a restorative tooth. An improved bond according to the present disclosure may provide a decrease in the chance of a failure between the temporary coping and the restorative tooth as compared to a conventional mechanical bond.

As used herein, the term “chemical bond” refers to a chemical bond between the material of a temporary coping and the material of the restorative tooth. A chemical bond according to the present teachings refers to a direct bond between the materials forming the chemical bond, for example, without an adhesive between the two materials and without relying on a mechanical engagement of cooperating structures to form the bond. Materials that are chemically bonded are fused together by molecular bonds.

The present disclosure contemplates various exemplary embodiments for a coping, such as a temporary coping, that provides a bond to a restorative tooth, such as a temporary tooth. The temporary coping according to various exemplary embodiments of the present disclosure may form a chemical bond with the restorative tooth. In various exemplary embodiments, the bond may provide an improvement over conventional bonds that rely on, for example, mechanical features and/or an adhesive bonding material, such as, for example, cement.

Various exemplary temporary copings of the present disclosure comprise a material capable of forming a chemical bond with a restorative tooth. For example, the material of the temporary coping may form a chemical bond with the material of the restorative tooth. For example, the material of the temporary coping may form a chemical bond with the material of the restorative tooth as the tooth is molded around the temporary coping.

While not wishing to be bound by theory, it is believed that chemical bonds formed between the temporary coping and restorative tooth may be stronger than the mechanical bonds achieved by conventional temporary coping-tooth pairings, including the use of adhesive agents, such as cement. In at least some exemplary embodiments, the improved bond between the temporary coping and the restorative tooth may allow for the design and use of smaller temporary copings, which in turn, may allow for greater design flexibility in the restorative tooth. Smaller temporary copings may be possible because of the increased strength of the bond, as well as the elimination of space required for adhesive agents, such as cement. For example, a smaller temporary coping may allow a smaller tooth to be fabricated for a patient, which may provide better fit, better comfort, and better performance of the tooth. In addition, the improved bond between the temporary coping and the tooth may reduce the chance of failure of the temporary restoration.

In at least one exemplary embodiment, the temporary coping comprises a material capable of forming a chemical bond with a restorative tooth that is chosen from bis-acrylic or a bis-acrylic composite, also known as a bis-acrylic hybrid. In at least one embodiment of the present disclosure, the bis-acrylic or bis-acrylic composite comprises ProTemp™ Plus Temporization Material available from 3M ESPE.

In at least one exemplary embodiment of the present disclosure, the temporary coping comprises a bis-acrylic composite. Examples of bis-acrylic composites that may be used according to the present disclosure include but are not limited to, for example, Luxatemp®, a composite of bis-acrylic, glass powder, and silica, available from DMG; InstaTemp® Max, a composite of bis-acrylic, glass powder, and silica, available from Sterngold Dental; Structur Premium, Acytemp, Integrity Fluorescence, and Kanitemp Royal.

In at least one exemplary embodiment of the present disclosure, the temporary coping comprises a material capable of forming a chemical bond with a restorative tooth and having a material toughness (i.e., fracture work) of at least about 10.0 KJ/m² (Kilo-Joules per square meter). The material toughness is a measure of the amount of energy that may be absorbed by the material before it fractures, and may be measured by any method known to those of skill in the art.

In at least one exemplary embodiment of the present disclosure, the temporary coping comprises a material capable of forming a chemical bond with a restorative tooth and having a fracture toughness of at least about 1.0 MPa*m /^{1/2} (Mega-Pascal per meter^{1/2}). In at least one further embodiment, the temporary coping comprises a material capable of forming a chemical bond with a restorative tooth and having a fracture toughness of at least 1.5 MPa*m^{1/2}, such as at least 2.0 MPa*m^{1/2}. The fracture toughness is a measure of a material's resistance to crack propagation when a crack is present, and may be measured by any method known to those of skill in the art.

Temporary copings in accordance with at least one exemplary embodiment of the present disclosure comprise a material capable of forming a chemical bond with a restorative tooth and having a compressive strength of at least about 350 MPa. In at least one further embodiment, the material capable of forming a chemical bond with a restorative tooth has a compressive strength of at least about 375 MPa. The compressive strength is a measure of a material's resistance to compression, and may be measured by any method known to those of skill in the art.

As discussed above, bis-acrylic or bis-acrylic composites may exhibit the various material toughness, fracture toughness, and/or compressive strength properties noted above.

The restorative tooth may comprise a material that may be chemically bonded to the temporary coping disclosed in the various embodiments of the present disclosure. Materials that may form chemical bonds include, for example, various polymers and composites. Bis-acrylic and bis-acrylic composites are exemplary polymers and composites that may form chemical bonds with temporary copings. One of ordinary skill in the art would readily appreciate that other materials capable of forming a chemical bond with temporary copings according to the present disclosure may be used for the restorative tooth.

In various exemplary embodiments, the temporary copings according to the present disclosure may optionally further comprise bonding mechanisms in addition to the chemical bond provided by the material of the temporary copings. For example, the temporary copings may comprise mechanical bonding aids, for example, surface features that provide a mechanical bond, or may comprise further chemical bonding aids.

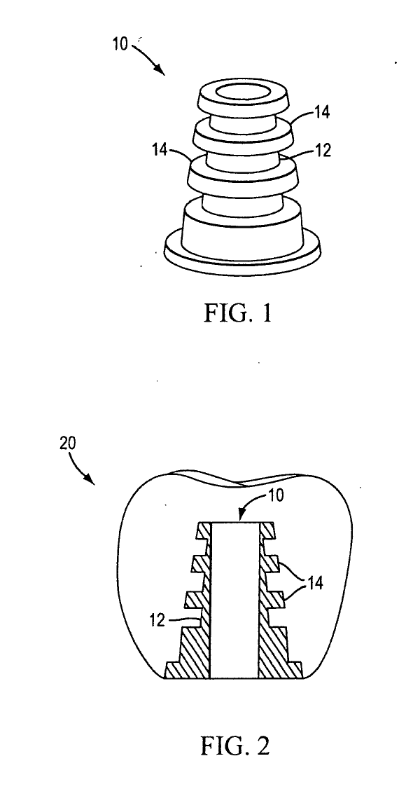

An exemplary embodiment of a temporary coping of the present disclosure comprising mechanical bonding aids, in addition to the chemical bonding provided by the material from which the temporary coping is made, is shown in FIG. 1. The temporary coping 10 is generally frustoconical in shape and has a hollow interior to fit on a provisional coping or an abutment provided on a dental implant through one end (not shown). The side 12 of temporary coping 10 comprises a plurality of retention rings 14 that provide a mechanical bond when a tooth is formed on temporary coping 10. FIG. 2 shows a schematic of a restorative tooth 20 formed on temporary coping 10. The material of tooth 20 surrounds temporary coping 10 and fills in the spaces around retention rings 14, thus mechanically binding tooth 20 to temporary coping 10 in addition to the chemical bonding provided by the materials of the tooth and the temporary coping. In the exemplary embodiment of FIGS. 1 and 2, retention rings 14 may increase the strength of the bond between the temporary coping and the tooth by providing mechanical bonding in addition to the chemical bonding between the temporary coping and tooth.

Although the exemplary embodiment of FIGS. 1 and 2 depict a series of retention rings 14 on an external surface of the temporary coping 10, those having ordinary skill in the art would understand that the retention rings shown are exemplary and non-limiting. For example, any number and/or arrangement of such retention rings may be provided without departing from the scope of the present teachings. In addition or in lieu of retention rings, other mechanical bonding surface features may be provided on the outer surface of the temporary coping over which the restorative tooth is placed. Such mechanical bonding surface features may include, but are not limited to, for example, roughened outer surface portions, one or more grooves, dimples, and/or other protrusions.

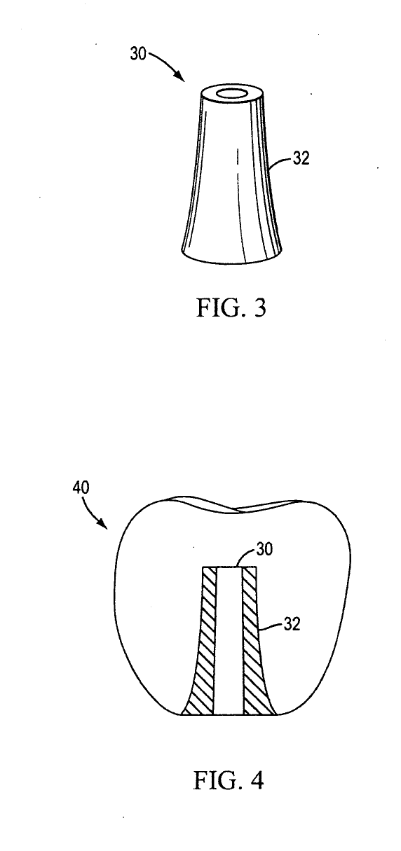

FIGS. 3 and 4 show an exemplary embodiment of a temporary coping according to the present disclosure that does not utilize mechanical bonding surface features. In FIG. 3, temporary coping 30 is generally frustoconical in shape and has substantially smooth sides 32 free of mechanical bonding surface features. FIG. 4 shows restorative tooth 40 on temporary coping 30 bound only by chemical bonding between the tooth and the temporary coping. In the exemplary embodiment shown in FIGS. 3 and 4, the temporary coping is bonded to the restorative tooth by chemical bonding only as a result of the material from which the temporary coping 30 is made.

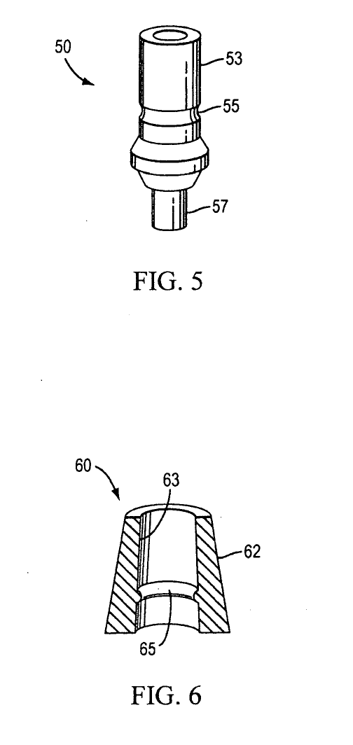

In at least one exemplary embodiment of the present invention, the temporary coping may be attached to a provisional coping. A provisional coping and a cutaway of a corresponding temporary coping according to at least one exemplary embodiment are shown in FIG. 5 and FIG. 6, respectively. In FIG. 5, the exemplary provisional coping 50 comprises a tapered or cylindrical mating portion 53, which is designed to mate to temporary coping 60, shown in FIG. 6. Groove 55 on mating portion 53 provides a mechanical bond to raised annular ring 65 on an interior surface 63 of temporary coping 60. Temporary coping 60 further comprises a frustoconical outer surface 62. A restorative tooth may be attached to outer surface 62. Provisional coping 50 may comprise, for example, titanium, and have a mating portion 57 to join the provisional coping to a dental implant. Temporary coping 60 may comprise a material capable of forming a chemical bond with a restorative tooth, such as, for example, bis-acrylic or a bis-acrylic composite that is capable of chemically bonding to a bis-acrylic or bis-acrylic composite restorative tooth.

The temporary copings according to exemplary embodiments of the present disclosure may comprise any shape or form useful for the intended application. The figures provided show various exemplary geometries and configurations. However, one ordinarily skilled in the art would understand that copings having different taper angles, either smaller or larger, are also contemplated by the present disclosure. Furthermore, other mechanical bonding aids, such as grooves, surface roughening, friction fitting features, or bumps, may be formed on the outer surface of the coping. In addition, the coping may comprise other structural features. For example, the coping may comprise protrusions, ridges, or bumps on an interior surface of the coping to aid in the binding of the coping to a provisional coping or an abutment. Such deviations from the structures shown in the figures are contemplated by the present disclosure, and one ordinarily skilled in the art would readily appreciate that such structural modifications are covered by the present disclosure. Those ordinarily skilled in the art would appreciate that a variety of coping configurations may be made of the materials described herein in order to provide a coping configured to form a chemical bond with restorative tooth in accordance with the present teachings.

The coping according to the present disclosure may be made by any known process, including, for example, injection molding, extrusion, casting, compression molding, transfer molding, machining, or cutting. For example, the material, such as liquid bis-acrylic or bis-acrylic composite, may be injected into a mold to fabricate the coping. In another exemplary embodiment, the coping may be formed by cutting and shaping a solid piece of material.

The present disclosure also contemplates methods for forming a bond between a restorative tooth and a coping. In at least one exemplary embodiment of the present disclosure, the bond between a restorative tooth and a coping may be improved by forming a chemical bond between the tooth and the coping.

In at least one exemplary embodiment of the present invention, the bond between the restorative tooth and the coping may be formed by selecting a coping and tooth made of a material capable of forming a chemical bond. In at least one exemplary embodiment, the material capable of forming a chemical bond is chosen from a bis-acrylic and/or a bis-acrylic composite, or hybrid. According to one exemplary embodiment of the present disclosure, the restorative tooth may be formed on the coping by a process of injection molding. The restorative tooth may comprise liquid bis-acrylic or bis-acrylic composite which may, for example, be injected around a coping, such as a bis-acrylic or bis-acrylic composite coping, in a mold. As the injected material cures, or hardens, a bond forms between the tooth and the bis-acrylic or bis-acrylic composite of the coping. After the restorative tooth is formed, it may be shaped to fit around the patient's gumline and surrounding dentition. When bis-acrylic or a bis-acrylic composite is used to form the coping and restorative tooth, shrinkage that occurs when bis-acrylic or bis-acrylic composites cure may also improve the bond between the coping and the restorative tooth. The shrinkage of the bis-acrylic or bis-acrylic composite may create a compression fit.

The present disclosure also contemplates methods forming a bond between a coping and a restorative tooth comprising the steps of providing a coping comprising a material capable of forming a chemical bond to a restorative tooth, fabricating a restorative tooth over the coping, and curing the restorative tooth. In at least one exemplary embodiment, the material capable of forming a chemical bond to a restorative tooth may be chosen from bis-acrylic and a bis-acrylic composite.

It is noted that, as used in this specification and the appended claims, the singular forms “a,” “an,” and “the,” include plural referents unless expressly and unequivocally limited to one referent. Thus, for example, reference to “a retention ring” can refer to one, two, or more retention rings. As used herein, the term “include” and its grammatical variants are intended to be non-limiting, such that recitation of items in a list is not to the exclusion of other like items that can be substituted or added to the listed items.

It will be apparent to those skilled in the art that various modifications and variation can be made to the copings and methods of the present disclosure without departing from the scope its teachings. Other exemplary embodiments of the disclosure will be apparent to those skilled in the art from consideration of the specification and practice of the teachings disclosed herein. It is intended that the embodiments described in the specification be considered as exemplary only.

What is claimed is:

1. A temporary coping for placement as an intermediate structure between a temporary restorative tooth and a support on a dental implant, the temporary coping comprising:
a coronal end, an apical end, and a lateral wall surrounding a hollow interior, wherein the hollow interior is dimensioned to receive a support mountable on a dental implant to place the temporary coping in mating engagement with the support along a substantial length of the hollow interior;
wherein the temporary coping is made of a dental restoration material chosen from bis-acrylic and a bis-acrylic composite and is chemically bondable with the temporary restorative tooth.

2. The temporary coping of claim 1, wherein the bis-acrylic composite comprises bis-acrylic, silica, and glass.

3. The temporary coping of claim 1, wherein the dental restoration material has a material toughness of at least about 10.0 KJ/m².

4. The temporary coping of claim 1, wherein the dental restoration material has a fracture toughness of at least about 1.0 MPa*m^{1/2}.

5. The temporary coping of claim 4, wherein the dental restoration material has a fracture toughness of at least about 1.5 MPa*m^{1/2}.

6. The temporary coping of claim 1, wherein the dental restoration material has a compressive strength of at least about 350 MPa.

7. The temporary coping of claim 6, wherein the dental restoration material has a compressive strength of at least about 375 MPa.

8. The temporary coping of claim 1, further comprising at least one mechanical bonding surface feature on an outer surface of the temporary coping.

9. The temporary coping of claim 8, wherein the at least one mechanical bonding surface feature comprises at least one retention ring.

10. The temporary coping of claim 1, wherein the apical end comprises an opening leading to the hollow interior.

11. The temporary coping of claim 1, wherein the temporary coping is configured for mating engagement with a tapered or cylindrical support.

12. A method for temporary dental implant restoration, the method comprising:
forming a temporary restorative tooth on a temporary coping, wherein the temporary coping is made of a dental restoration material chosen from bis-acrylic and a bis-acrylic composite and comprises a coronal end, an apical end, and a lateral wall surrounding a hollow interior dimensioned to receive a support structure mountable on a dental implant;
allowing the temporary restorative tooth to harden and chemically bond to the temporary coping;
securing the temporary coping to the support structure; and
after an elapsed period of time, removing the temporary coping and the temporary restorative tooth from the support structure.

13. The method of claim 12, wherein the dental restoration material is chosen from a bis-acrylic composite comprising bis-acrylic, silica, and glass.

14. The method of claim 12, wherein the dental restoration material has a material toughness of at least about 10.0 KJ/m².

15. The method of claim 12, wherein the dental restoration material has a fracture toughness of at least about 1.0 MPa*m^{1/2}.

16. The method of claim 15, wherein the dental restoration material has a fracture toughness of at least about 1.5 MPa*m^{1/2}.

17. The method of claim 12, wherein the dental restoration material has a compressive strength of at least about 350 MPa.

18. The method of claim 17, wherein the dental restoration material has a compressive strength of at least about 375 MPa.

19. The method of claim 12, wherein forming the temporary restorative tooth comprises injection molding the restorative tooth onto the temporary coping.

20. The method of claim 12, wherein the securing of the temporary coping to the support structure comprises inserting the support structure through the apical end of the temporary coping into the hollow interior of the temporary coping.
